(19) United States
(12) Patent Application Publication
(10) Pub. No.: US 2022/0313397 A1
(43) Pub. Date: Oct. 6, 2022

(54) PERIODONTAL TISSUE REGENERATION INDUCER AND APPARATUS AND METHOD FOR MANUFACTURING THE SAME

(71) Applicant: NANOBIOSYSTEM Co., LTD., Gwangju (KR)

(72) Inventors: Kyoung Jin SHIN, Gwangju (KR); Sung Ho KIM, Gwangju (KR)

(73) Assignee: NANOBIOSYSTEM Co., LTD., Gwangju (KR)

(21) Appl. No.: 17/848,987

(22) Filed: Jun. 24, 2022

Related U.S. Application Data
(62) Division of application No. 16/823,038, filed on Mar. 18, 2020.

(30) Foreign Application Priority Data
Feb. 25, 2020 (KR) 10-2020-0023014

(51) Int. Cl. A61C 8/02; B29C 59/02; B29C 59/00

(52) U.S. Cl. CPC A61C 8/0006; B29C 59/022; B29C 59/002; A61C 2201/00; B29K 2067/04

(57) ABSTRACT

A periodontal tissue regeneration inducer and an apparatus and a method for manufacturing the same are provided. The periodontal tissue regeneration inducer includes a sheet body formed of a flexible material, and a pattern layer having a pattern formed therein to facilitate bone formation or regeneration on a surface of the sheet body. Therefore, the periodontal tissue regeneration inducer and the apparatus and method for manufacturing the same have an advantage in that the bone formation or regeneration may be facilitated during implant surgery because the membrane sheet is composed of a polycaprolactone (PCL) material and a nanopattern is formed on a surface of the pattern layer.

### CROSS-REFERENCE TO RELATED APPLICATION

This application is a divisional application of U.S. patent application Ser. No. 16/823,038 filed on Mar. 18, 2020, which claims priority to and the benefit of Korean Patent Application No. 10-2020-0023014, filed Feb. 25, 2020, the disclosure of which is incorporated herein by reference in its entirety.

### BACKGROUND

### 1. Field of the Invention

The present invention relates to a periodontal tissue regeneration inducer and an apparatus and a method for manufacturing the same, and more particularly, to a periodontal tissue regeneration inducer having a pattern formed therein so that bone formation or regeneration is easily induced on a surface of a membrane sheet, and an apparatus and a method for manufacturing the same.

### 2. Discussion of Related Art

Dental implant surgery is a surgery performed by cutting a patient's gum, inserting a dental implant (i.e., an artificial tooth root) into an alveolar bone present inside the gum, and coupling an artificial crown to the inserted dental implant.

It is a known fact that, when the dental implant is placed in the alveolar bone, the presence of sufficient bone surrounding the dental implant enhances an initial fixation force of the dental implant and improves surgical prognosis. However, the presence of insufficient bone surrounding the dental implant often causes bone defects, which results in poor prognosis after placement of the dental implant.

Therefore, to solve the above problem, a membrane is used for periodontal surgery for the purpose of inducing tissue and bone regeneration. The membrane hinders translocation of regenerated tissue into other sites and penetration of epithelial cells into the regenerated tissue for the time required to regenerate tissue and bone.

However, the membrane has a drawback in that it is impossible to completely hinder the penetration of other epithelial cells or suppress the propagation of bacteria. There are various proposed prior-art techniques to solve this drawback.

As one of the proposed prior-art techniques, Korean Patent Laid-Open Publication No. 10-2014-0061233 discloses an absorbable collagen membrane and a method of preparing the same. The method includes preparing collagen, mixing a natural polymeric material with the collagen, and cross-linking the natural polymeric material, and has an advantage in that it is possible to realize an absorbable collagen membrane which has excellent tensile strength and may spontaneously decompose or may be absorbed into the periodontal tissue after a certain period of time.

However, the prior-art document has some drawbacks in that a membrane sheet is easily attached to the bone, and materials of the membrane sheet and a pattern on a surface of the membrane sheet are different from those of the membrane sheet according to the present invention. Also, there are some differences in a specific configuration of the membrane sheet and in the method of preparing the membrane sheet.

### PRIOR-ART DOCUMENT

### Patent Document

Patent Document 1: Korean Patent Laid-Open Publication No. 10-2014-0061233: Absorbable Collagen Membrane and Method of Preparing the Same

### SUMMARY OF THE INVENTION

The present invention is designed to solve the problems of the prior art, and therefore it is an object of the present invention to provide a periodontal tissue regeneration inducer, which consists of a sheet body and a pattern layer having a pattern formed therein to facilitate bone formation or regeneration, and an apparatus and a method for manufacturing the same.

To achieve the above object, according to one aspect of the present invention, there is provided a periodontal tissue regeneration inducer which includes a sheet body formed of a flexible material, and a pattern layer having a pattern formed therein to facilitate bone formation or regeneration on a surface of the sheet body.

The sheet body and the pattern layer are composed of a polycaprolactone (PCL) material and have a nanopattern formed on a surface thereof.

Meanwhile, the method for manufacturing a periodontal tissue regeneration inducer according to one exemplary embodiment of the present invention includes: a coating step of coating glass with a polymer to a predetermined thickness, a pattern molding step of molding a pattern by pressing the coated polymer with a patterned mold, and a separation step of separating the polymer from the glass after the pattern molding step.

The coating step may include putting the polymer on the glass and spin-coating the glass while rotating the glass.

The method of the present invention may further include a planarization step of planarizing a surface of the coated polymer between the coating step and the pattern molding step, wherein the planarization step includes: a primary heating step of heating the coated polymer, a primary in-mold molding step of molding the heated polymer by pressing a surface of the heated polymer with a flat mold, and a primary cooling step of cooling the molded polymer.

The pattern molding step may include: a secondary heating step of heating the coated polymer, a secondary in-mold molding step of molding the heated polymer by pressing the heated polymer with a patterned mold, and a secondary cooling step of cooling the molded polymer.

The primary cooling step may include cooling the planarized polymer by spraying air onto the glass.

The separation step may further include separating the polymer from the glass by immersing the glass coated with the polymer in ethanol, and a drying step of drying the separated polymer.

According to another aspect of the present invention, there is provided an apparatus for manufacturing a periodontal tissue regeneration inducer, which includes a frame, a pattern molding unit installed on the frame to move in a vertical direction and configured to mold a pattern by pressing the polymer with which the glass is coated, a support unit installed below the pattern molding unit and configured to support the glass and heat and cool the polymer in order to mold the polymer, and a transfer unit configured to transfer the support unit to face the pattern molding unit.

The pattern molding unit may include a first molding part configured to planarize the polymer, and a second molding part configured to mold a pattern on the planarized polymer.

Each of the first molding part and the second molding part may include an actuator, a mold coupling part formed to move in a vertical direction by the actuator, and a molding mold provided in the mold coupling part, and the first molding mold of the first molding part may have a flat surface formed therein, and the second molding mold of the second molding part may have a nanopattern formed on a surface thereof.

The mold coupling part may include a coupling plate coupled to the actuator, and a mold fixing member detachably installed in the coupling plate, having a mold hole formed therein to expose a surface of the molding mold to the polymer, and coupled to the coupling plate to support the molding mold.

The support unit may include a support plate, a hot plate installed on the support plate and configured to supply heat, and an air supply part installed at the support plate and configured to supply air in order to cool the polymer.

The transfer unit may include a guide rail, and a driving part driven along the guide rail while supporting the support plate.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a diagram showing a periodontal tissue regeneration inducer according to one preferred embodiment of the present invention;

FIG. 2 is a block diagram showing a method for manufacturing a periodontal tissue regeneration inducer according to one preferred embodiment of the present invention;

FIG. 3 is a conceptual diagram showing the method for manufacturing a periodontal tissue regeneration inducer according to one preferred embodiment of the present invention;

FIG. 4 is a perspective view showing an apparatus for manufacturing a periodontal tissue regeneration inducer according to one preferred embodiment of the present invention;

FIG. 5 is a front view showing the apparatus for manufacturing a periodontal tissue regeneration inducer according to one preferred embodiment of the present invention;

FIG. 6 is a partially exploded perspective view showing the apparatus for manufacturing a periodontal tissue regeneration inducer according to one preferred embodiment of the present invention;

FIG. 7 is an exploded perspective view showing a pattern molding unit and a support unit of the apparatus for manufacturing a periodontal tissue regeneration inducer according to one preferred embodiment of the present invention; and

FIG. 8 is an exploded perspective view showing a coupling plate and a mold fixing member according to one preferred embodiment of the present invention.

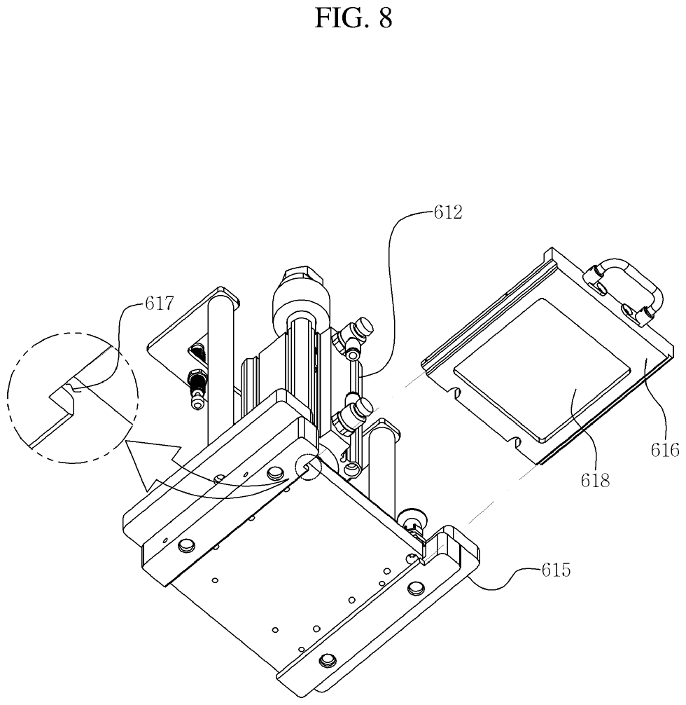

### DETAILED DESCRIPTION OF EXEMPLARY EMBODIMENTS

Hereinafter, “periodontal tissue regeneration inducer and apparatus and method for manufacturing the same” according to one exemplary embodiment of the present invention will be described in detail with reference to the accompanying drawings. The present invention may be modified into various forms and may have various embodiments, and specific embodiments thereof will be illustrated in the drawings and described herein in detail. However, it should be understood that the description set forth herein is not intended to limit the particular embodiments of the present invention, and encompasses all modifications, equivalents, and substitutions that fall within the spirit and scope of the present invention. Throughout the description of the figures, like numbers refer to like elements. In the accompanying drawings, the dimensions of parts are shown to be more exaggerated than they actually are for clarity of the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of the present invention.

The terminology used herein is for the purpose of describing particular embodiments only and is not intended to limit the present invention. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which this invention belongs. It will be further understood that the terms, such as those defined in commonly used dictionaries, should be interpreted as having a meaning that is consistent with their meaning in the context of the relevant art and will not be interpreted in an idealized or overly formal sense unless expressly so defined herein.

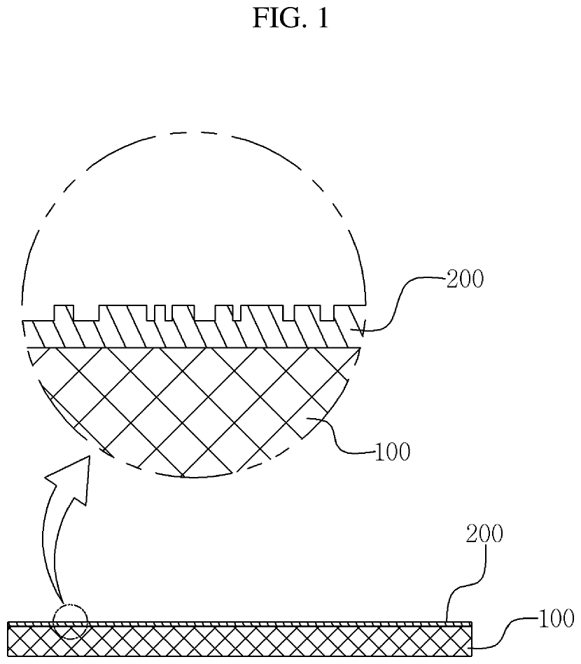

Referring to FIG. 1, a periodontal tissue regeneration inducer according to one exemplary embodiment of the present invention includes a sheet body 100 and a pattern layer 200.

The sheet body 100 is preferably formed of a flexible material so that the sheet body 100 can be attached to the bone during implant surgery. The pattern layer 200 has a pattern formed therein to facilitate bone formation or regeneration on a surface of the sheet body 100.

The sheet body 100 and pattern layer 200 are composed of a polycaprolactone (PCL) material and have a nanopattern formed on a surface thereof. According to one exemplary embodiment of the present invention, the PCL material is applied, but the present invention is not limited thereto. For example, various materials such as polyglycolic acid (PGA), polylactic acid (PLA), poly-lactic-co-glycolic acid (PLGA), poly(L-lactic acid) (PLLA), and the like may be applied as the PCL material.

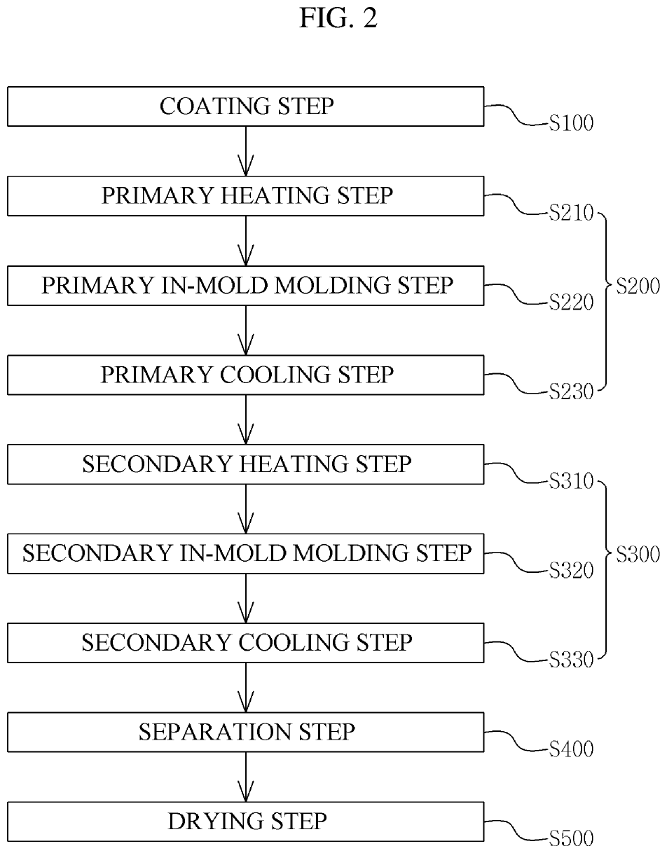

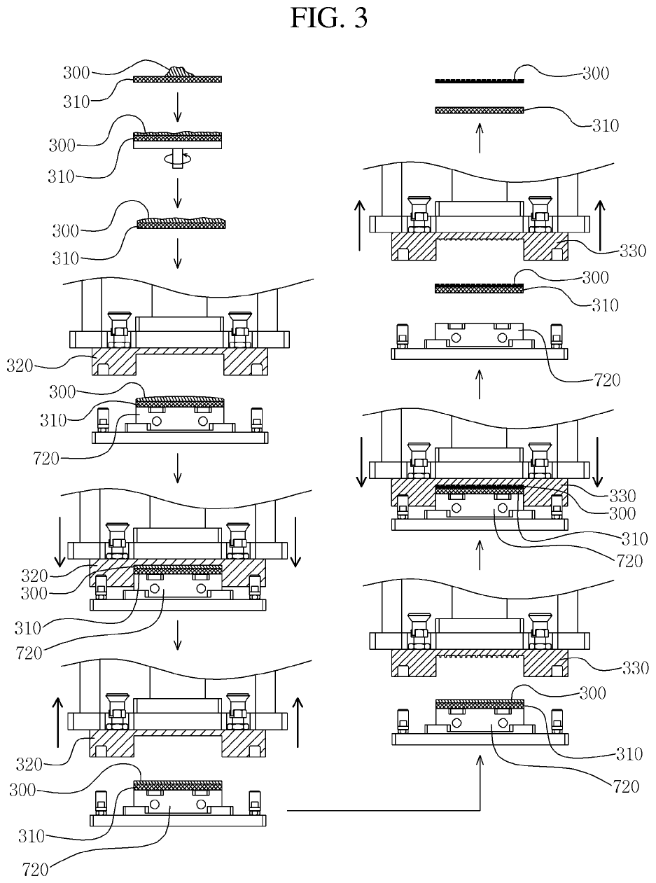

Referring to FIGS. 2 and 3, a method for manufacturing a periodontal tissue regeneration inducer according to one exemplary embodiment of the present invention includes a coating step S100, a planarization step S200, a pattern molding step S300, a separation step S400, and a drying step S500.

In the coating step S100, glass 310 is coated with a polymer 300 to a predetermined thickness. In the coating step S100, the polymer 300 is put on the glass 310, and the glass 310 is then spin-coated while rotating the glass 310.

In the planarization step S200, a surface of the polymer is planarized. For this purpose, the planarization step S200 includes a primary heating step S210, a primary in-mold molding step S220, and a primary cooling step S230.

In the primary heating step S210, the coated polymer 300 is heated. In the primary in-mold molding step S220, the polymer 300 heated in the primary heating step S210 is molded by pressing a surface of the polymer 300 with a flat mold 320.

In the primary cooling step S230, the polymer 300 is cooled so that the polymer whose surface is planarized by pressing with the flat mold 320 can be separated from the mold. In the primary cooling step S230, the planarized polymer 300 is cooled by spraying air onto the glass 310.

In the pattern molding step S300, a pattern is molded by pressing the coated polymer 300 with a patterned mold 330. For this purpose, the pattern molding step S300 includes a secondary heating step S310, a secondary in-mold molding step S320, and a secondary cooling step S330.

In the secondary heating step S310, the coated polymer 300 is heated. In the secondary in-mold molding step S320, the heated polymer 300 is molded by pressing the heated polymer 300 with the patterned mold 330. In this case, the pattern preferably consists of a nanopattern. In the secondary cooling step S330, the molded polymer 300 is cooled.

In the separation step S400, the polymer 300 is separated from the glass 310 after the pattern molding step S300. In the separation step S400, the polymer 300 is separated from the glass 310 by immersing the glass 310 coated with the polymer 300 in ethanol.

In the drying step S500, the polymer 300 immersed in the ethanol is dried. In the drying step S500, the polymer 300 is dried by natural drying. Finally, a periodontal tissue regeneration inducer of the present invention is manufactured.

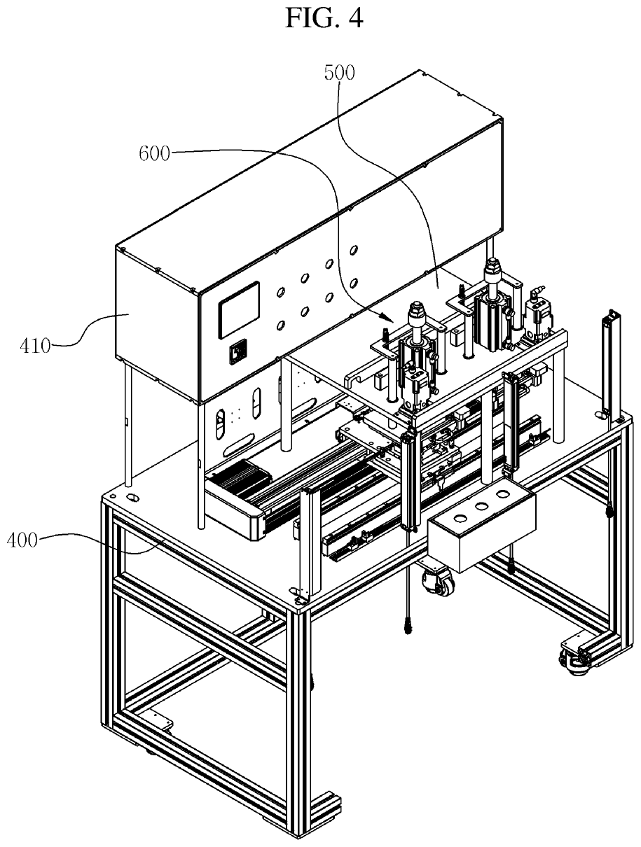

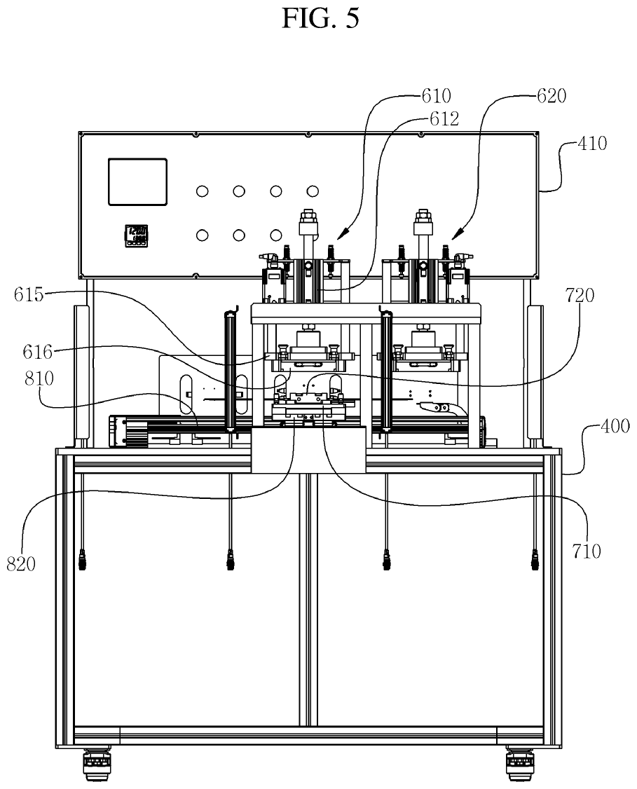

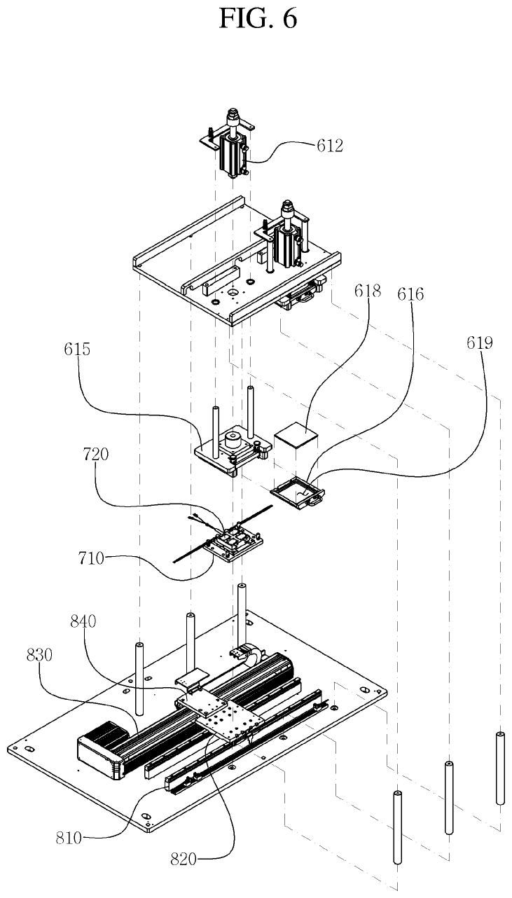

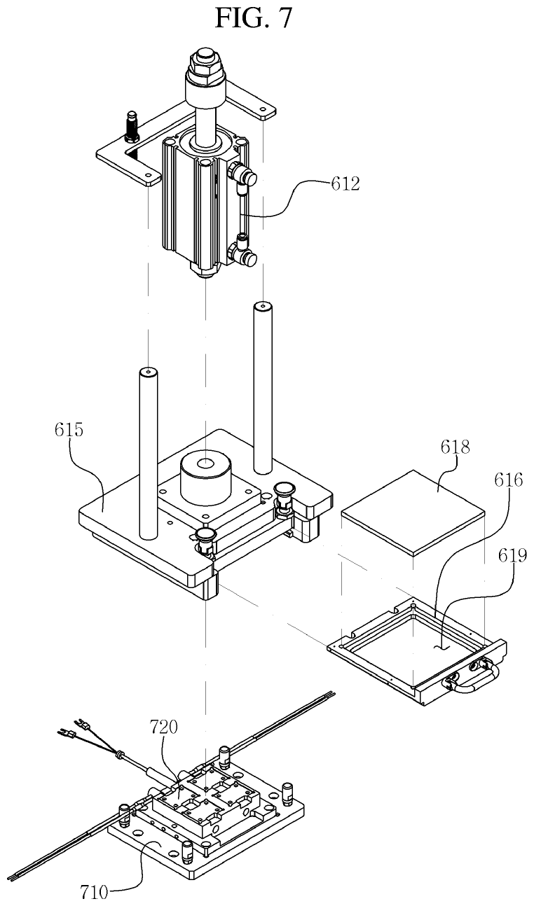

Referring to FIGS. 4 to 7, an apparatus 10 for manufacturing a periodontal tissue regeneration inducer according to one exemplary embodiment of the present invention includes a holder 400, a frame 500, a pattern molding unit 600, a support unit 700, and a transfer unit 800.

The holder 400 is configured to allow a user to manipulate and control the apparatus 10 for manufacturing a periodontal tissue regeneration inducer. In this case, a control panel 410 is provided on one side of the holder 400. The frame 500 is installed on the holder 400, and is then formed so that the frame 500 is coupled to the pattern molding unit 600.

The pattern molding unit 600 is configured to mold a pattern by pressing the polymer 300 with which the glass 310 is coated. In this case, the pattern molding unit 600 is installed on the frame 500 to move in a vertical direction, and includes a first molding part 610 and a second molding part 620.

The first molding part 610 is configured to planarize polymer 300 on the glass 310. The second molding part 620 is configured to mold a pattern onto the planarized polymer 300, and is installed side by side with the first molding part 610 so that the second molding part 620 is adjacent to the first molding part 610.

Each of the first molding part 610 and the second molding part 620 includes an actuator 612, a mold coupling part 614, and a molding mold 618.

The actuator 612 is a pneumatic cylinder that operates with hydraulic pressure or pneumatic pressure, and is generally configured to provide a mechanical operation so that the pressure is applied to the polymer 300, and thus a detailed description thereof is omitted for clarity.

The mold coupling part 614 is formed to move in a vertical direction by the actuator 612, and includes a coupling plate 615 and a mold fixing member 616.

The coupling plate 615 is coupled to the actuator 612, and has a sliding groove 617 formed therein.

The mold fixing member 616 has a mold hole 619 formed therein to expose a surface of the molding mold 618 to the polymer 300, and is coupled to the coupling plate 615 to support the molding mold 618. Therefore, the mold fixing member 616 is detachably installed in the coupling plate 615. The mold fixing member 616 is attachable or detachable because the mold fixing member 616 is formed to be pulled in/drawn out along the sliding groove 617 of the coupling plate 615.

The molding mold 618 is provided in the mold coupling part 614. The first molding mold 618 of the first molding part 610 has a flat surface formed therein. A nanopattern is formed on a surface of the second molding mold 618 of the second molding part 620. The molding mold 618 according to one exemplary embodiment of the present invention has a flat surface or has a nanopattern formed on a surface thereof, but the present invention is not limited thereto. For example, patterns with various shapes are applicable to the molding mold 618.

The support unit 700 is installed below the pattern molding unit 600, and is configured to support the glass 310 and heat and cool the polymer 300 in order to mold the polymer 300. For this purpose, the support unit 700 includes a support plate 710, a hot plate 720, and an air supply part.

The support plate 710 is configured to support the hot plate 720. In this case, the support plate 710 is preferably made of a resin having high durability to withstand the heat generated by the hot plate 720.

The hot plate 720 is installed with the glass 310 coated with the polymer 300, and is configured to indirectly supply a heat source to the glass 310. In this case, the hot plate 720 is installed on the support plate 710.

Although not shown in the drawings, the air supply part is configured to supply air in order to cool the polymer 300 with which the glass 310 is coated, and includes an air nozzle and a connection hose.

The air nozzle is installed at the support plate 710, and is configured to spray air onto the hot plate 720 or the glass 310 coated with the polymer 300. The connection hose is connected to the air nozzle to supply air.

Therefore, the air flowing from the outside through the connection hose is supplied to the air nozzle. Then, the supplied air is sprayed through the air nozzle to cool the hot plate 720 or the glass 310. As a result, the polymer 300 is hardened.

The transfer unit 800 is configured to transfer the support unit 700 to face the pattern molding unit 600, and includes a guide rail 810, a driving part 820, a fixed rail 830, and a fixed driving part 840.

The guide rail 810 is formed to extend in a horizontal direction. In this case, one or more guide rails are installed on the holder 400 to face the first molding part 610 and the second molding part 620 of the pattern molding unit 600. A metal material is preferably used in the guide rail 810.

The driving part 820 is driven along the guide rail 810. In this case, the driving part 820 is configured to support the support plate 710 and is installed to be driven along the guide rail 810.

One or more fixed rails 830 are installed on the holder 400 to be side by side with the guide rail 810. The fixed driving part 840 is configured to fix the driving part 820, and is driven along the fixed rail 830. Also, the fixed driving part 840 is arranged to face the driving part 820, and is configured to fix the driving part 820.

An operation of the apparatus 10 for manufacturing a membrane sheet according to one exemplary embodiment of the present invention is as follows.

First, when a user installs the glass 310 coated with the polymer 300 on the hot plate 720, the polymer 300 is melted by the hot plate 720. At the same time, the driving part 820 is driven along the guide rail 810 to face the first molding part 610.

In this case, the polymer 300 is planarized by pressing the polymer 300 arranged on the polymer 300 with the first molding mold 618 of the first molding part 610. At the same time, after an operation of the hot plate 720 of the support unit 700 is stopped, the molten polymer 300 arranged on the glass 310 is hardened, and the first molding part 610 is spaced apart from the polymer 300. As a result, this planarization process is completed.

Then, the hardened polymer 300 on the glass 310 is melted by the hot plate 720 of the support unit 700. At the same time, the driving part 820 is driven along the guide rail 810 to face the second molding part 620.

In this case, a nanopattern is formed by pressing the polymer 300 arranged on the glass 310 with the second molding mold 618 of the second molding part 620. At the same time, after an operation of the hot plate 720 is stopped, the molten polymer 300 arranged on the glass 310 is hardened, the second molding part 620 is spaced apart from the polymer 300, and a nanopattern is formed on the polymer 300. The polymer 300 on which the nanopattern is formed is finally separated from the glass 310. As a result, the periodontal tissue regeneration inducer according to the present invention is manufactured.

The periodontal tissue regeneration inducer according to the present invention and the apparatus and method for manufacturing the same have an advantage in that bone formation or regeneration cab be facilitated during implant surgery because the membrane sheet is composed of a polycaprolactone (PCL) material and a nanopattern is formed on the pattern layer.

The description of the presented embodiments is provided so that those skilled in the art to which the present invention belongs use or implement the present invention. It will be apparent to those skilled in the art that various changes and modifications can be made to these embodiments, and that general principles defined herein can be applied to other embodiments without departing from the scope of the present invention. Therefore, the present invention is not limited to the embodiments presented herein, but should be interpreted within the widest range which is associated with the principles and new features presented herein.

[Brief Description of Main Parts in the Drawings]

```
  10: apparatus for manufacturing a periodontal tissue regeneration
  100: sheet body
  200: pattern layer
  300: polymer
  310: glass
  320: flat mold
  330: patterned mold
  400: holder
  410: control panel
  500: frame
  600: pattern molding unit
  610: first molding part
  612: actuator
  614: mold coupling part
  615: coupling plate
  616: mold fixing member
  617: sliding groove
  618: molding mold
  619: mold hole
  620: second molding part
  700: support unit
  710: support plate
  720: hot plate
  800: transfer unit
  810: guide rail
  820: driving part
  830: fixed rail
  840: fixed driving part
S100: coating step
S200: planarization step
S210: primary heating step
S220: primary in-mold molding step
S230: primary cooling step
S300: pattern molding step
S310: secondary heating step
S320: secondary in-mold molding step
S330: secondary cooling step
S400: separation step
S500: drying step
```

What is claimed is:

1. A method for manufacturing a periodontal tissue regeneration inducer, comprising:
a coating step of coating glass with a polymer to a predetermined thickness;
a pattern molding step of molding a pattern by pressing the coated polymer with a patterned mold; and
a separation step of separating the polymer from the glass after the pattern molding step.

2. The method of claim 1, wherein the coating step comprises putting the polymer on the glass and spin-coating the glass while rotating the glass.

3. The method of claim 1, further comprising a planarization step of planarizing a surface of the coated polymer between the coating step and the pattern molding step,
wherein the planarization step comprises:
a primary heating step of heating the coated polymer;
a primary in-mold molding step of molding the heated polymer by pressing a surface of the heated polymer with a flat mold; and
a primary cooling step of cooling the molded polymer, and
wherein the pattern molding step comprises:
a secondary heating step of heating the coated polymer;
a secondary in-mold molding step of molding the heated polymer by pressing the heated polymer with a patterned mold; and
a secondary cooling step of cooling the molded polymer.

4. The method of claim 3, wherein the primary cooling step comprises cooling the planarized polymer by spraying air onto the glass.

5. The method of claim 1, wherein the separation step further comprises:
separating the polymer from the glass by immersing the glass coated with the polymer in ethanol, and
a drying step of drying the separated polymer.
